(19) United States
(12) Patent Application Publication
(10) Pub. No.: US 2024/0078929 A1
(43) Pub. Date: Mar. 7, 2024

(54) SYSTEM FOR MONITORING WATER CONSUMPTION OF USERS AND A METHOD THEREOF

(71) Applicant: Mirza Faizan, Irving, TX (US)

(72) Inventors: Mirza Faizan, Irving, TX (US); Mustafa Hasani, Murphy, TX (US); Malak Askar, Southlake, TX (US); Adam Mhal, Plano, TX (US); Karim Medhat Askar, Southlake, TX (US); Manha Sadarulanam, Plano, TX (US); Sooryavanshi Narayanan, Frisco, TX (US); Meher Saanvi Singh, Southlake, TX (US); Jiya Saanvi Singh, Southlake, TX (US); Mirza Rizwan, Patna (IN); Benjamin Stafford, Dallas, TX (US); Sparsh Kamdar, Plano, TX (US); Vishy Narayanan, Frisco, TX (US); Abdullah Hasani, Murphy, TX (US)

(21) Appl. No.: 17/900,905

(22) Filed: Sep. 1, 2022

(51) Int. Cl. G09B 19/00

(52) U.S. Cl. CPC G09B 19/0092; A61B 5/117

(57) ABSTRACT

The present disclosure envisages a system and a method for monitoring water consumption of at least one user. The system comprises a water-holder unit configured to detect a registered user and further configured to measure an overall water consumption value of said registered user for a pre-defined time. The system further comprises a cloud server configured to receive said overall water consumption value of said registered user, perform analysis by employing at least one of artificial intelligence technique on said overall water consumption value of said registered user and generate a suggestive report depicting an actionable intelligence based on said analysis and a set of pre-defined parameters related to said registered user. The system stores in a repository data corresponding to said registered users, pre-defined time and a set of pre-defined parameters related to said registered user.

### FIELD

The present disclosure relates to the field of health-related monitoring devices. More specifically, the invention belongs to the field of health-related monitoring devices that monitor water consumption of users on the basis of health and other user-related parameters.

### BACKGROUND

In the era of the internet and intelligent devices, health monitoring devices are becoming common tools for monitoring the health of humans. These devices measure and monitor the vitals of the human body that helping the user and medical professionals to keep track of a healthy lifestyle and avoid diseases beforehand.

A conventionally known monitoring system includes one or more wireless nodes and an electromyography (EMG) sensor coupled to the person and the wireless nodes to determine a stroke attack. Similarly, there are other monitoring systems and devices that measure EEG, ECG, and so forth to determine strokes and attacks.

Other ranges of monitoring devices are seen in wearable devices that monitor vital signs and provide the user with monitoring results. Recent advances in the field are that users can see statistics related to these vital signs individually on the display of said wearable devices.

The conventional monitoring systems and devices are limited to real-time vital signs monitoring. Moreover, none of these systems indicate patterns followed by the human body.

Further, these devices and systems are designed to accommodate only dramatic diseases like heart attack, brain stroke, etc. The conventional systems do not focus on the basic needs of the human body like the amount of water consumption by the user.

There is, therefore, felt a need for a system and a method for monitoring water consumption of users based on various important factors related to users.

### Objects

Some of the objects of the present disclosure, which at least one embodiment herein satisfies, are as follows:

An object of the present disclosure is to provide a system and a method for monitoring the water consumption of users.

Another object of the present disclosure is to provide a system and a method that monitor water consumption based on various important factors related to users.

Still another object of the present disclosure is to provide a system and a method that generate a suggestive report for the water consumption of users.

Yet another object of the present disclosure is to provide a system and a method that performs the predictive analysis.

A further object of the present disclosure is to provide a system and a method that is inexpensive.

A still further object of the present disclosure is to provide a system and a method that assists users through the predictive report of water consumption.

An object of the present disclosure is to provide a system and a method that performs analysis based on artificial intelligence.

Another object of the present disclosure is to provide a system and a method that is easy to use.

A further object of the present disclosure is to provide a system and a method that is economical.

A still further object of the present disclosure is to provide a system and a method that employs machine learning for accuracy.

Other objects and advantages of the present disclosure will be more apparent from the following description, which is not intended to limit the scope of the present disclosure.

### SUMMARY

The present invention envisages a system for monitoring the water consumption of at least one user. The system comprises a water-holder unit configured to detect a registered user and further configured to measure an overall water consumption value of the registered user for a pre-defined time.

The system comprises a cloud server, communicatively coupled to the water-holder unit through a communication network, configured to receive the overall water consumption value of the registered user. The cloud server performs analysis by employing at least one of artificial intelligence technique on the overall water consumption value of the registered user and generates a suggestive report depicting an actionable intelligence based on the analysis and a set of pre-defined parameters related to the registered user.

A repository is also included in the system that is configured to store data corresponding to the registered users, pre-defined time, and a set of pre-defined parameters related to the registered user.

In an embodiment, the water-holder unit includes a touch sensor configured to receive inputs from at least one user.

In another embodiment, the pre-defined time is selected by the registered user from pre-stored options including hourly, daily, weekly, monthly, yearly, and a combination thereof.

The cloud server comprises a receiving module configured to receive the inputs from the touch sensor of the water-holder unit corresponding to at least one user. The cloud server includes a registration module configured to identify the registered user based on data received through the touch sensor and further configured to invoke a registration process in case of an unidentified user.

The system further includes a monitoring module configured to monitor the overall water consumption value of the user for the pre-defined time. The system further includes an analysis module configured to receive the monitored overall water consumption value corresponding to each of the registered users and further configured to analyze the overall water consumption value of the registered user by employing at least one artificial intelligence technique to generate an actionable intelligence related to the water consumption of each of the user.

The system includes a report generation module configured to receive the actionable intelligence data to generate the suggestive report based on the analysis and the set of pre-defined parameters related to the registered user.

In an embodiment, the monitoring module is further configured to monitor the overall water consumption value by determining the difference between the first total volume of water inside the water-holder unit filled initially and the second volume of water left inside the water-holder unit in various instances of time.

In another embodiment, the monitoring module is further configured to determine the overall water consumption value by adding differences determined above.

In yet another embodiment, the pre-defined parameters include information related to height, weight, BMI, heart rate, blood pressure, oxygen level, daily activity, workout routine, disorders, diseases, work hours, sleep hours, and stress level of said registered user.

In still another embodiment, the suggestive report includes a predictive analysis and a general analysis.

In a further embodiment, the predictive analysis is generated weekly, monthly, quarterly, half-yearly, or annually based on a pattern detected through the history of pre-defined parameters inputted by the registered user.

In a still further embodiment, the general analysis is generated hourly or daily based on pre-defined parameters inputted by the registered user every day for alerting the user to improve water consumption.

A method for monitoring water consumption of at least one user, the method comprising steps of:
receiving inputs from at least one user;
detecting a registered user or invoking a registration process in case of an unidentified user;
monitoring an overall water consumption value of the registered user based on a pre-defined time;
receiving the monitored overall water consumption value corresponding to each of the registered users;
analysing the monitored overall water consumption value of the registered user by employing at least one of artificial intelligence technique to generate an actionable intelligence related to the water consumption of each of the users; and
generating a suggestive report based on the analysis and the set of pre-defined parameters related to the registered user.

These and other features, advantages, and objects of the various embodiments will be better understood with reference to the following drawings, specifications, and claims.

### BRIEF DESCRIPTION OF THE ACCOMPANYING DRAWING

The accompanying drawings, which are included to provide a further understanding of the invention and are incorporated in and constitute a part of this specification, illustrate embodiments of the invention and together with the description serve to explain the principles of the invention.

The following figure depicts a certain illustrative embodiment of the invention. This depicted embodiment is to be understood as illustrative of the invention and not as limiting in any way.:

FIG. 1 illustrates an exemplary network environment for monitoring water consumption of users, in accordance with an embodiment of the present disclosure;

FIG. 2 illustrates an exemplary functional block diagram of the cloud server of FIG. 1; and

FIG. 3 illustrates an exemplary method flow diagram for monitoring the water consumption of users, in accordance with an embodiment of the present disclosure.

### LIST OF REFERENCE NUMERALS

100—System
102—Water-holder unit
104—Cloud server
106—Communication network
108—Repository
110—Interface
112—Processor
114—Memory
202—Receiving module
204—Registration module
206—Monitoring module
208—Analysis module
210—Report generation module
212—Configuration data
214—Device & application data
216—User related data
218—Other data

### DETAILED DESCRIPTION

Embodiments are provided so as to thoroughly and fully convey the scope of the present disclosure to the person skilled in the art. Numerous details are set forth relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The following detailed description illustrates the invention by way of example, and not by way of limitation. This description will clearly enable one skilled in the art to make and use the invention and describes several embodiments, adaptions, variations, alternatives, and uses of the invention, including what we presently believe is the best mode of carrying out the invention.

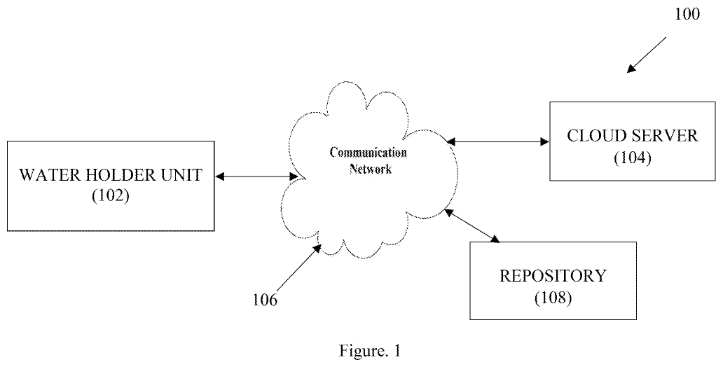

The present invention envisages a system and a method for monitoring the water consumption of at least one user. The present invention will now be described with reference to FIG. 1 of the preferred embodiment of the present invention.

The present invention envisages a system 100 for monitoring the water consumption of at least one user.

System 100 can be an ‘loT’ based system. ‘loT’ here refers to the Internet of things, which is an interconnection of various computing devices via the internet in everyday objects. This interconnection enables connected devices to send and receive data from each other.

The system 100 for monitoring water consumption of at least one user, where the system comprises a water-holder unit 102, a cloud server 104, and a repository 108.

The system 100 is not limited to only components 102, 104, and 108 as communication between 102, 104, and 108 is provided by means of a communication network 106. Network connectivity can be wired or wireless between 102, 104, and 108.

However, wireless connectivity is preferable for the convenience of the user. Examples of wireless connectivity between 102, 104, and 108 can be selected from Bluetooth, infrared and other types that support devices in proximity to connect.

In an embodiment, the communication network 106 may include, but is not limited to, a direct interconnection, an e-commerce network, a Peer-to-Peer (P2P) network, Local Area Network (LAN), Wide Area Network (WAN), wireless network (for example, using Wireless Application Protocol), Internet, Wi-Fi and the like.

The water-holder unit 102 is configured to detect a registered user. Once the identity of the user is detected the water-holder unit 102 measures an overall water consumption value of the registered user for a pre-defined time.

In an embodiment, the pre-defined time is selected by the registered user from pre-stored options including hourly, daily, weekly, monthly, yearly, and a combination thereof.

For example, the user can select amongst abovesaid options and input the monitoring schedule for closely monitoring the water consumption in a day or a week.

In an embodiment, the water-holder unit 102 includes a touch sensor configured to receive inputs from the user. The touch sensor allows for the identification of the user as a validated user to use the system upon touch by the user.

The cloud server 104 is communicatively coupled with the water-holder unit through the communication network 106.

The cloud server 104 upon receiving input from the water holder unit 102 identifies whether the user is registered with system 100 or not. In case the user is a new user, the cloud server 104 provides a web form on the display of water-holder unit 102. Through the web form, a new user can fill in the details required by the system 100, and a profile corresponding to user inputs is created in the cloud server 104.

The cloud server 104, after validating the identity of the user, receives the overall water consumption value of the registered user.

The cloud server 104 now performs analysis by employing at least one artificial intelligence technique on the overall water consumption value of the registered user and generate a suggestive report depicting an actionable intelligence based on the analysis and a set of pre-defined parameters related to said registered user.

The examples of actionable intelligence may include, drinking more water every hour, consuming electrolytes along with the water, consuming a lesser quantity of water per week, etc.

The suggestive report is based on the analysis and a set of pre-defined parameters related to the registered user.

In an embodiment, the pre-defined parameters include information related to height, weight, BMI, heart rate, blood pressure, oxygen level, daily activity, workout routine, disorders, diseases, work hours, sleep hours, and stress level of the registered user.

These pre-defined parameters are inputted by the users at the beginning step of registration. Also, the user can update the details if required through the display of water-holder unit 102.

The vitals of the user's body can be measured using various sensors known in the art. Examples of sensors can be heart rate monitoring sensors, pressure sensors, oximeters, etc.

Repository 108 of the system 100 is configured to store data corresponding to the registered users, pre-defined time, and a set of pre-defined parameters related to the registered user.

The cloud server 104 further includes a crawler and extractor to fetch information and data from repository 108 whenever desired.

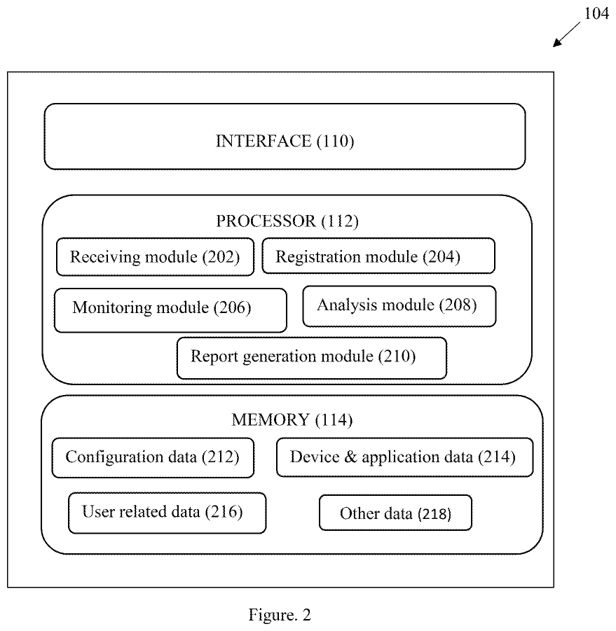

The present invention will now be described with reference to FIG. 2 of the preferred embodiment of the present invention.

The cloud server 104 further comprises a receiving module 202, a registration module 204, a monitoring module 206, an analysis module 208, and a report generation module 210.

The receiving module 202 is configured to receive the inputs from the touch sensor of the water-holder unit corresponding to at least one user. The input may include a pattern of lines of the finger.

The registration module 204 receives the pattern of finger and identifies whether the user is registered with the system 100 or not. In case the user is not registered with the system 100, a registration process is invoked by the registration module 204.

The registration module 204 transmits a web form to the display of the water-holder unit 102. Through the web form, a new user can fill in the details required by the system 100, and a profile corresponding to user inputs is created in the cloud server 104.

After the user is registered and authenticated, the monitoring module 206 receives the overall water consumption value of the registered user. The monitoring module 206 monitors the overall water consumption value of the user for the pre-defined time. This pre-determined time is selected by the user itself based on his own needs.

In an embodiment, the pre-defined time is selected by the registered user from pre-stored options including hourly, daily, weekly, monthly, yearly, and a combination thereof.

For example, the user can select amongst abovesaid options and input the monitoring schedule for closely monitoring the water consumption monthly for better suggestions on intake of water.

In an embodiment, the monitoring is performed by determining a difference between a first total volume of water inside the water-holder unit 102 filled initially and the second volume of water left inside water-holder unit 102 in various instances of time.

Further, the differences obtained at different instances for a user, suppose say user A, are added to calculate the overall water consumption value.

The analysis module 208 is configured to receive the overall water consumption value corresponding to each of the registered users. The analysis module 208 is further configured to analyze the overall water consumption value of the registered user by employing at least one artificial intelligence technique to generate an actionable intelligence related to the water consumption of each of the users.

In an embodiment, the artificial intelligence techniques are selected from the group consisting of predictive analysis techniques and time series analysis techniques.

In another embodiment, the artificial intelligence techniques employed include, but are not limited to, Naïve Bayes, decision trees, linear regression, neural networks, random forest, and the like.

The report generation module 210 is configured to receive the actionable intelligence data to generate the suggestive report based on the analysis and the set of pre-defined parameters related to the registered user.

In an embodiment, the suggestive report includes a predictive analysis and a general analysis. The predictive analysis is generated weekly, monthly, quarterly, half-yearly, or annually based on a pattern detected through the history of pre-defined parameters inputted by the registered user.

In another embodiment, the general analysis is generated hourly or daily based on pre-defined parameters inputted by the registered user every day for alerting the user to improve water consumption.

The cloud server 104 can be implemented by one or more processors or a processor 110 that may be a general-purpose processor, a digital signal processor, an application-specific integrated circuit, a field-programmable gate array or another programmable logic device, a discrete gate or transistor logic device, or a discrete hardware component, and can implement or perform the methods, steps, and logical block diagrams disclosed in the embodiments of this invention.

The general-purpose processor may be a microprocessor or any conventional processor or the like. The steps of the method disclosed with reference to the embodiments of this application may be directly performed by a hardware processor or may be performed by using a combination of hardware in the processor and a software unit.

The cloud server 104 further includes an I/O interface 110, a memory 114, and the processor 112. The I/O interface 110 may be configured to receive data from the water-holding unit 102.

The data from the I/O interface 110 may be stored in memory 114. The memory 114 may be communicatively coupled to the processor 112 that executes all the modules 202 to 210.

The memory 114 may also store processor instructions which may cause the processor 110 to execute the instructions for managing continuous execution of the modules.

In another preferred embodiment, various type of data is stored in the cloud server 104 that may include, for example, configuration data 212, device and application data 214, user-related data 216, and other data 218.

The configuration data 212 may include configuration information associated with the cloud server 104. The configuration data 212 may contain a list of registered users and user devices. In an embodiment, the list is updated when a new user device is registered.

The device and application data 214 may include device configuration information such as device identification number, device type, and supported application identification number, used application details such as, comprises application identifier application type, application state, and application restriction.

The user-related data 216 may include registered users' information such as user identification number, date, time, the location associated with the devices, login ID, password, card details, etc.

The other data 218 may store data, including temporary data and temporary files, generated by modules for performing the various functions of the cloud server 104.

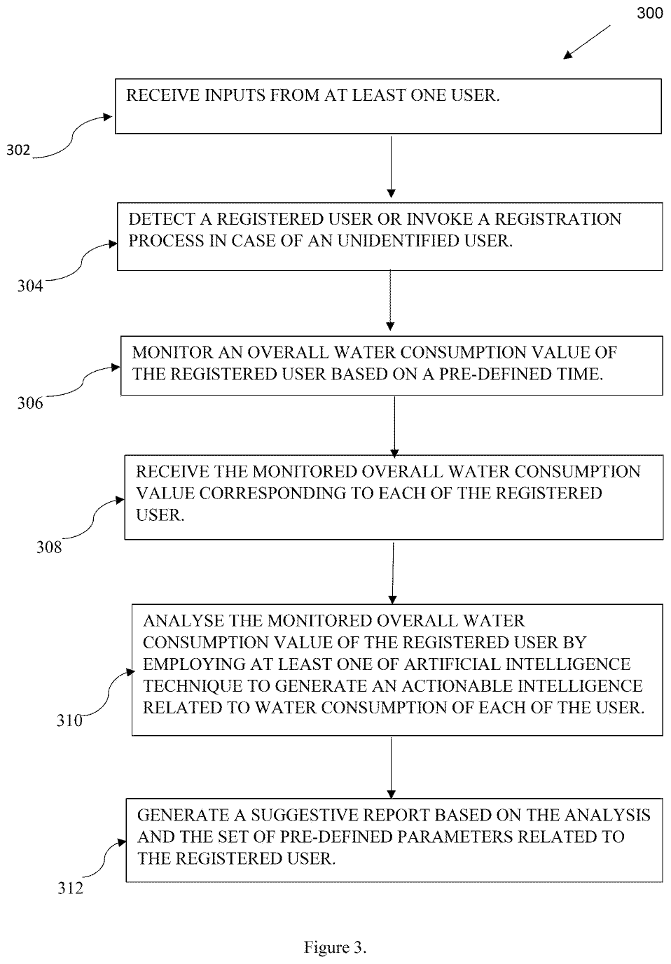

FIG. 3 will now be described that illustrates a method 300 for monitoring water consumption of users according to a preferred embodiment of the present invention.

Method 300 comprises the step of receiving 302 inputs from at least one user. The method 300 further includes the step of detecting 304 a registered user or invoking a registration process in case of an unidentified user.

Upon registration and authentication of the user, the method 300 performs the step of monitoring 306 an overall water consumption value of the registered user based on a pre-defined time.

The method 300 further includes receiving 308 the monitored overall water consumption value corresponding to each of the registered user and analyzing 310 the monitored overall water consumption value of the registered user by employing at least one artificial intelligence technique to generate an actionable intelligence related to the water consumption of each of the user.

The method 300 now performs the step of generating 312 a suggestive report based on the analysis and the set of pre-defined parameters related to the registered user.

According to the system and method disclosed herein, a system and method in accordance with the present invention provide numerous benefits. It provides a useful, novel, and non-trivial solution to the problem of monitoring the water consumption of users.

Although the present invention has been described in accordance with the embodiments shown, one of ordinary skill in the art will readily recognize that there could be variations to the embodiments and those variations would be within the spirit and scope of the present invention.

Accordingly, many modifications may be made by one of ordinary skill in the art without departing from the spirit and scope of the appended claims.

### Technical Advancements

The present disclosure described herein above has several technical advantages including, but not limited to, the realization of a system and a method for monitoring water consumption of users that:
monitors water consumption of users based on hours, months, years;
monitor water consumption based on various important factors related to users;
generate a suggestive report for water consumption of users;
performs predictive analysis and results into inexpensive setup;
assists users through the predictive report of water consumption;
performs analysis based on artificial intelligence;
is easy to use;
is economical; and
employs machine learning for accuracy.

What is claimed is:

1. A system for monitoring water consumption of at least one user, said system comprising:
a water-holder unit configured to:
detect a registered user; and
measure an overall water consumption value of said registered user for a pre-defined time;
a cloud server, communicatively coupled to said water-holder unit through a communication network, wherein said cloud server is configured to:
receive said overall water consumption value of said registered user;
perform analysis by employing at least one of artificial intelligence technique on said overall water consumption value of said registered user; and
generate a suggestive report depicting an actionable intelligence based on said analysis and a set of pre-defined parameters related to said registered user; and
a repository configured to store data corresponding to said registered users, pre-defined time and a set of pre-defined parameters related to said registered user.

2. The system for monitoring water consumption as claimed in claim 1, wherein said water-holder unit includes a touch sensor configured to receive inputs from said at least one user.

3. The system for monitoring water consumption as claimed in claim 1, wherein said pre-defined time is selected by said registered user from pre-stored options including hourly, daily, weekly, monthly, yearly and a combination thereof.

4. The system for monitoring water consumption as claimed in claim 2, said cloud server comprises:
a receiving module configured to receive said inputs from said touch sensor of said water-holder unit corresponding to said at least one user;
a registration module configured to:
identify said registered user based on data received through said touch sensor; and
invoke a registration process in case of an unidentified user;
a monitoring module configured to monitor said overall water consumption value of said user for said pre-defined time;
an analysis module configured to:
receive said monitored overall water consumption value corresponding to each of said registered user; and
analyse overall water consumption value of said registered user by employing at least one of artificial intelligence technique to generate an actionable intelligence related to water consumption of each of said user; and
a report generation module configured to receive said actionable intelligence data to generate said suggestive report based on said analysis and said set of pre-defined parameters related to said registered user.

5. The system for monitoring water consumption as claimed in claim 1, wherein said monitoring module is further configured to:
monitor said overall water consumption value by determining difference between a first total volume of water inside said water-holder unit filled initially and a second volume of water left inside water-holder unit in various instances of time.

6. The system for monitoring water consumption as claimed in claim 5, wherein said monitoring module is further configured to determine said overall water consumption value by adding said determined differences.

7. The system for monitoring water consumption as claimed in claim 1, wherein said pre-defined parameters include information related to height, weight, BMI, heart rate, blood pressure, oxygen level, daily activity, workout routine, disorders, diseases, work hours, sleep hours and stress level of said registered user.

8. The system for monitoring water consumption as claimed in claim 1, wherein said suggestive report includes a predictive analysis and a general analysis.

9. The system for monitoring water consumption as claimed in claim 1, wherein:
said predictive analysis is generated weekly, monthly, quarterly, half-yearly or annually based on a pattern detected through history of pre-defined parameters inputted by said registered user; and
said general analysis is generated hourly or daily based on pre-defined parameters inputted by said registered user every day for alerting user to improve water consumption.

10. A method for monitoring water consumption of at least one user, said method comprising steps of:
receiving inputs from said at least one user;
detecting a registered user or invoking a registration process in case of an unidentified user;
monitoring an overall water consumption value of said registered user based on a pre-defined time;
receiving said monitored overall water consumption value corresponding to each of said registered user;
analysing said monitored overall water consumption value of said registered user by employing at least one of artificial intelligence technique to generate an actionable intelligence related to water consumption of each of said user; and
generating a suggestive report based on said analysis and said set of pre-defined parameters related to said registered user.
